(19) United States
(12) Patent Application Publication
(10) Pub. No.: US 2003/0087913 A1
(43) Pub. Date: May 8, 2003

(54) Solid pharmaceutical agent formulation for a piperazine urea derivative

(75) Inventors: Heiko Kranz, Berlin (DE); Christoph Volkel, Jena (DE); Ralph Lipp, Berlin (DE); Johannes Tack, Berlin (DE); Herbert Wiesinger, Berlin (DE)

Correspondence Address: MILLEN, WHITE, ZELANO & BRANIGAN, P.C., 2200 CLARENDON BLVD., SUITE 1400, ARLINGTON, VA, 22201, US

(73) Assignee: Schering AG, Berlin (DE)

(21) Appl. No.: 10/273,368

(22) Filed: Oct. 18, 2002

Related U.S. Application Data
(60) Provisional application No. 60/330,410, filed on Oct. 22, 2001.

(30) Foreign Application Priority Data
Oct. 18, 2001 (DE) 10152351.3

(51) Int. Cl. A61K 9/14; A61K 31/495

(52) U.S. Cl. 514/255.01; 424/486

(57) ABSTRACT

A solid pharmaceutical agent formulation that contains (2R)-1-((4-chloro-2-(ureido)phenoxy)methyl)carbonyl-2-methyl-4-(4-fluorobenzyl)piperazine or a salt thereof is described.

This application claims the benefit of the filing date of U.S. Provisional Application Serial No. 60/330,410 filed Oct. 22, 2001.

The invention relates to a solid pharmaceutical agent formulation that contains (2R)-1-((4-chloro-2-(ureido)phenoxy)methyl)carbonyl-2-methyl-4-(4-fluorobenzyl)piperazine or a salt thereof.

WO 98/56771 describes benzylpiperazine urea compounds and especially (2R)-1-((4-chloro-2-(ureido)phenoxy)methyl)carbonyl-2-methyl-4-(4-fluorobenzyl)-piperazine and its salts. These substances are antagonists of the CCR-1 receptor and are used in the treatment of inflammatory diseases, i.a., multiple sclerosis and rheumatoid arthritis. In addition, they are used in psoriasis and atopic dermatitis. They are very poorly soluble at basic pH values. At a pH of 1, about 5 mg/ml is dissolved from (2R)-1-((4-chloro-2-(ureido)phenoxy)methyl)carbonyl-2-methyl-4-(4-fluorobenzyl)piperazine hydrogen sulfate, while at a pH of 6.35 or 6.8, only about 0.15 mg/ml or 0.1 mg/ml in each case is dissolved. Owing to this very poor solubility in the intestinal tract, no therapeutically necessary uniform plasma levels can be reached, while avoiding significant side effects, in the case of conventional oral formulation. In addition to the increase in solubility in the intestinal tract, it would be desirable, moreover, that the release of the active ingredient be carried out in a controlled manner over an extended period so that dosage intervals can be significantly extended. At the same time, however, an industrial-scale production of the medication also had to be possible.

In the literature, various methods to increase the absorption of poorly soluble active ingredients have been described (e.g., in “Techniques of Solubilization of Drugs,” S. H. Yalkowsky Ed. in Drugs and the Pharmaceutical Sciences). The use of solubilizers, such as, e.g., surfactants for very poorly soluble substances (WO01/05376), is especially recommended. This method was only poorly suitable, however, for solving this problem. The addition of the surfactant SDS to (2R)-1-((4-chloro-2-(ureido)phenoxy)methyl)carbonyl-2-methyl-4-(4-fluorobenzyl)-piperazine hydrogen sulfate resulted only in a slight increase in the release (see FIG. 2).

Other publications deal with the problem of pH-independent release. Streubel et al. (2000, J Controlled Release 67, 101-110) describe the addition of acids to a pharmaceutical substance. The described pharmaceutical substance is very well dissolved, however, even without the addition of acids at a pH of 6.35 (more than 100 mg/ml). The goal of Streubel et al. was to offset the pH-induced fluctuations. This was achieved by the addition of acids. With this invention, the problem is to offset not only pH-induced fluctuations but also to increase the solubility per se. The properties of the pharmaceutical substance described by Streubel are distinguished significantly from those of this active ingredient (2R)-1-((4-chloro-2-(ureido)phenoxy)methyl)carbonyl-2-methyl-4-(4-fluorobenzyl)piperazine. Moreover, the formulation is used only for individual production of tablets, not for large-scale production. It was therefore uncertain whether this method can be used for the problem underlying the invention.

This invention solves the problem of increasing solubility and the pH-independent release with simultaneous industrial producibility by a solid pharmaceutical agent formulation that contains (2R)-1-((4-chloro-2-(ureido)phenoxy)methyl)carbonyl-2-methyl-4-(4-fluorobenzyl)piperazine or a salt thereof, whereby the pharmaceutical agent formulation in addition contains a polymer matrix, an organic acid and one or more adjuvants for directed control of the pH-independent pharmaceutical substance release (release modification) and for influencing the mechanical strength of the dosage forms, and the particle sizes of the powder mixtures are up to 90% in the range between 0.1 and 750 μm.

(2R)-1-((4-Chloro-2-(ureido)phenoxy)methyl)carbonyl-2-methyl-4-(4-fluorobenzyl)piperazine is referred to as piperazine urea below and has the following structure:

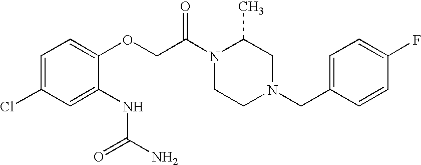

The production of (2R)-1-((4-chloro-2-(ureido)phenoxy)methyl)carbonyl-2-methyl-4-(4-fluorobenzyl)piperazine and its salts is carried out according to the method that is described in WO98/56771 in Example 2.

Salts thereof are, e.g., hydrochloride, dihydrogen phosphate, hydrogen sulfate, sulfate, mesylate, ethyl sulfonate, malate, fumarate and tartrate.

Solid pharmaceutical agent formulations in terms of the invention are single-unit systems, such as, e.g., tablets, and multiparticulate systems. Multiparticulate systems can be, e.g., granular grains, pellets or minitablets. The latter can be filled in hard or soft gelatin capsules and can be pressed into tablets. In most cases, the original shaped body dissolves in the stomach into many subunits. The mini-depots then overflow successively from the stomach into the intestine. In this case, the mini-depots can generally pass into the pylorus if the sphincter is closed.

A polymer matrix can be selected from the group that consists of cellulose derivatives [e.g., methyl cellulose, hydroxypropyl methyl cellulose, (e.g., hydroxypropyl methyl cellulose K 4 M, hydroxypropyl methyl cellulose K 15 M), hydroxypropyl cellulose, hydroxyethyl cellulose, sodium-carboxy methyl cellulose, ethyl cellulose (e.g., ethyl cellulose 100), cellulose acetate (e.g., cellulose acetate CA-398-10 NF), cellulose acetate phthalate, cellulose acetate propionate, cellulose acetate butyrate (e.g., cellulose acetate butyrate 171-15 PG), cellulose butyrate, cellulose nitrate, hydroxypropyl methyl cellulose phthalate, hydroxypropyl methyl cellulose acetate succinate]; acryl derivatives [e.g., polyacrylates, cross-linked polyacrylates (e.g., polymethacrylates, polyethylacrylates, polymethylic acid ethyl acrylates, polymethylic acid methyl methacrylates, polymethylic acid methyl methacrylates, polymethylacrylate trimethylammonium ethyl methacrylate chlorides, polyethylacrylate trimethylammonium ethyl methacrylate chlorides, dimethylaminoethyl methacrylate methacrylate copolymers, Carbopol® 971 P, Carbopol® 974 P, Carbopol® 71 G)], vinyl polymers (e.g., polyvinyl pyrrolidones, polyvinyl acetates, polyvinyl acetate phthalates), polyethylene glycols, polyanhydrides, polyester polyorthoesters, polyurethanes, polycarbonates, polyphosphazenes, polyacetals, polysaccharides (e.g., xanthans, xanthan gum), sugar esters (e.g., saccharose stearate, saccharose palmitate, saccharose laurate, saccharose behenate, saccharose oleate, saccharose erucate and saccharose ester with mixed fatty acids), diethylene glycol-monoethyl ethers (e.g., Transcutol® P), diethylene glycol monopalmitostearate (e.g., Hydrine®), ethylene glycol monopalmitostearate (e.g., Monthyle®), glycerol behenates and glycerol dibehenates (e.g., Compritol® 888 ATO, Compritol® HD 5 ATO and Compritol® E), glycerol distearates, glycerol dipalmitostearates, and glycerol palmitostearates (e.g., Precirol® ATO 5 and Precirol® WL 2155), glycerol-monooleate 40 (e.g., Peceol®), glycerol-monostearate 40-55 (e.g., Geleol®), macrogolglycerol-laurates (e.g., Gelucire® 44/14 and Labrafil® M 2130 CS), macrogolglycerol-stearates (e.g., Gelucire® 50/13), propylene glycol-monopalmitostearate (e.g., Monosteol®), chitosan, galactomannan, pectin, shellac and alginates. Especially suitable is a physical mixture that consists of water-insoluble polyvinyl acetate and water-soluble polyvinyl pyrrolidone as a polymer matrix. This mixture, which in addition contains sodium lauryl sulfate and silicon dioxide, is marketed, e.g., under the trade name Kollidon SR® (Kollidon SR, Technical Information, ME 397e, BASF, July 2000: 80% polyvinyl acetate, 19% polyvinyl pyrrolidone, 0.8% sodium lauryl sulfate and 0.2% silicon dioxide).

The organic acid can be selected from the group that consists of fumaric acid, citric acid, trisodium citrate, Na-hydrogen citrate, ascorbic acid, maleic acid, maleic acid anhydride, tartaric acid, adipic,acid, Na-hydrogen phosphate, succinic acid, glutaric acid, glutaric acid anhydride, potassium sorbate and sorbic acid. Fumaric acid is preferred.

For directed control of the pH-independent pharmaceutical substance release (release modification) and for influencing the mechanical strength of the dosage form, water-soluble or else water-insoluble adjuvants, such as, e.g., lactose, calcium diphosphates, mannitol, sorbitol, saccharose, fructose, glucose, starch or a starch derivative can be used. Mixtures that consist of one or more adjuvants can also be used. Lactose is preferred. Especially advantageous is coarse-grained lactose.

As an additional adjuvant for directed control of the pH-independent pharmaceutical substance release (release modification) and for influencing the mechanical strength of the dosage form, cellulose or cellulose derivatives can be used. Especially advantageous is microcrystalline cellulose. The latter swells in an aqueous environment and results in an improved pH-independent release of the piperazine urea and its salts.

In addition, lubricants can be added to the single-unit dosage forms, such as, e.g., tablets, to reduce interparticulate friction and to reduce the sliding friction between the material and matrix wall. As lubricants, substances are used that, because of their lamellar structure, have layers that can be moved slightly against one another. Pharmaceutically usable organic substances are, e.g., the divalent metallic soaps, the higher fatty alcohols and the polyethylene glycols with higher molecular weights. Especially advantageous are the magnesium and calcium salts of higher fatty acids.

In the case of single-unit dosage forms, a flow-regulating agent can be added to improve the flow properties of the material to be put into tablet form. This has the result that the material to be put into tablet form fills the matrix of the machine uniformly with sufficient packing density. The addition of a flow-regulating agent can be necessary in particular in the case of direct tableting. Substances with a pure flow-regulating action are mainly the highly dispersed silicic acids, i.e., the micronized silica gels and the pyrolytically produced silicic acids. Starches and talc are substances that can be used as flow-regulating agents, as well as decomposition adjuvants or as lubricants.

In the case of the single-unit dosage form, it is important for its industrial-scale production that the material to be put into tablet form have granulate-like properties, such as good flowability, high bulk density and defined grain size distribution. The grain size of the material to be put into tablet form depends in this case on the size of the tablets to be produced and generally varies between 0.1-750 μm. Within the material to be put into tablet form, as uniform a grain size distribution as possible is important to prevent a separation (e.g., during vibrating of the tablet machine) and thus an accumulation of larger particles in the upper portion of the material, since otherwise greater fluctuations can occur in the dosage. A defined particle size and particle size distribution is achieved by classification (e.g., wet or dry sifting) or by granulation of the starting substances. The particle size can be measured with the aid of the process that is described in Example 5. The particle sizes should be up to 90% in the range between 0. 1-750 μm. A range of 20-400 μm is preferred.

The piperazine urea or its salts can be dispersed homogeneously in the matrix or be surrounded by the matrix. In the latter case, the active ingredient forms a core that is surrounded by the matrix shell.

The solid pharmaceutical agent formulation in terms of this invention can also be coated with a color lake to provide for optical and flavoring considerations. The latter generally consists of a binder (e.g., hydroxypropyl methyl cellulose, polyvinyl pyrrolidone, polyethylene glycol), lubricant (e.g., talc) and pigments (e.g., iron oxide pigment, titanium dioxide).

A preferred solid pharmaceutical agent formulation contains (2R)-1-((4-chloro-2-(ureido)phenoxy)methyl)carbonyl-2-methyl-4-(4-fluorobenzyl)piperazine or a salt thereof, lactose, Kollidon SR®, silicon dioxide and magnesium stearate, whereby 90% of the particles are in the range of 0.1-750 μm. Especially preferred is the use of hydrogen sulfate as a salt. A tablet with this formulation shows a 60% release of the piperazine urea after 6 hours.

Another preferred pharmaceutical agent formulation contains (2R)-1-((4-chloro-2-(ureido)phenoxy)methyl)carbonyl-2-methyl-4-(4-fluorobenzyl)piperazine or a salt thereof, microcrystalline cellulose, lactose, Kollidon SR®, silicon dioxide and magnesium stearate, whereby 90% of the particles are in the range of 0.1-750 μm. Especially preferred is the use of hydrogen sulfate as a salt. A tablet with this formulation shows an 80-90% release of the piperazine urea after 4 hours.

Another preferred solid pharmaceutical agent formulation contains (2R)-1-((4-chloro-2-(ureido)phenoxy)methyl)carbonyl-2-methyl-4-(4-fluorobenzyl)piperazine or a salt thereof, lactose, Kollidon SR®, silicon dioxide and magnesium stearate, whereby 90% of the particles are in the range of 0.1-750 μm, and the tablet then is coated with a color lake that consists of hydroxypropyl methyl cellulose, talc, titanium oxide and iron oxide pigment. A tablet with this formulation shows a 60% release of the piperazine urea after 6 hours.

The pharmaceutical agent formulation according to the invention considerably increases the solubility and the release of the piperazine urea and its salts. While in the case of a conventional formulation that consists of (2R)-1-((4-chloro-2-(ureido)phenoxy)methyl)carbonyl-2-methyl-4-(4-fluorobenzyl)piperazine or a salt thereof, lactose, corn starch, polyvinyl pyrrolidone, croscarmellose sodium and magnesium stearate, only about 10% is released after 8-10 hours at pH 6.8, the release is increased to about 60-90% by the formulation according to the invention. The advantage of the pharmaceutical agent formulation according to the invention is also shown in clinical studies. Compared with a conventional oral formulation, the plasma levels of (2R)-1-((4-chloro-2-(ureido)phenoxy)methyl)carbonyl-2-methyl-4-(4-fluorobenzyl)piperazine in the individuals being treated are increased when the formulation according to the invention is administered over a longer period (see FIG. 11).

The formulation according to the invention has all properties that are necessary for an industrial-scale production, such as, e.g., good flow properties, high bulk density, good dosage accuracy, high plastic deformability and thus slight compressibility and high mechanical strength of the tablets that are produced.

The subject matter of the invention is also a process for the production of a solid pharmaceutical agent formulation according to the invention, whereby (2R)-1-((4-chloro-2-(ureido)phenoxy)methyl)carbonyl-2-methyl-4-(4-fluorobenzyl)piperazine or a salt thereof is mixed with the polymer matrix, the organic acid, the lubricant and the adjuvant and is put into tablet form (direct tableting). The direct production of tablets is carried out in this case basically via a mixing of the powder components, a dosage via the filling device of the tablet machine, and subsequent compression of the powder mixing. In the case of direct tableting, the particle size and particle size distribution of the piperazine urea that is used and its salts, polymer matrix, organic acid and adjuvants have a considerable influence on the industrial-scale production of the tablets. The latter are therefore to be classified individually before the mixing of the powder components (e.g., by sieving). As an alternative, the entire powder mixture or individual components of the powder mixture can be classified together (e.g., sieved). The powder components are weighed, as mentioned in the Examples, and mixed over a sufficiently long period in a gravity mixer (e.g., turbula mixer, V-mixer) or forced-circulation mixer (e.g., plow blade mixer, planetary mixer-kneader). In particular, the flow-regulating agent and lubricant (both together are also referred to as an FST complex) are added only just shortly before the tableting machine is charged. In this case, the FST complex is to be finely,sieved onto the premixed tableting material and is to be admixed as described above, whereby the mixing time should be set neither too short (inhomogeneous distribution) nor too long (overmixing of the material).

In addition, the invention relates to a process for the production of a solid pharmaceutical agent formulation according to the invention, whereby (2R)-1-((4-chloro-2-(ureido)phenoxy)methyl)carbonyl-2-methyl-4-(4-fluorobenzyl)piperazine or a salt thereof, the polymer matrix, the organic acid and the adjuvant are subjected to a single operation that is referred to as granulation before the mixing and tableting. After the granulation and the addition of the lubricant, it is put into tablet form as described above. The granulation can be carried out in this case by step-by-step enlargement or agglomeration of primary particles of the powder mixture up to the desired secondary size (building granulation) or by division of a powder material that is made into a paste to the desired granulate grain size (decomposing granulation). The building granulation includes, e.g., the circular granulation and the fluidized-bed granulation. The decomposing granulation can be carried out by, e.g., compacting the starting substances and subsequent mechanical division and sieving of the compressed material. In this case, the decomposing or building granulation can be carried out wet (e.g., adhesive or crust granulates) or dry (e.g., briquette or melt-solidification granulates).

Another subject of the invention is a process for the production of a solid multiparticulate pharmaceutical agent formulation according to the invention, whereby (2R)-1-((4-chloro-2-(ureido)phenoxy)methyl)carbonyl-2-methyl-4-(4-fluorobenzyl)piperazine or a salt thereof, the polymer matrix, the organic acid and the adjuvant (preferably cellulose, cellulose derivatives, and lactose) are processed into pellets by means of extrusion and subsequent spheronization.

Another subject of the invention is a process for the production of a solid multiparticulate pharmaceutical agent formulation according to the invention, whereby (2R)-1-((4-chloro-2-(ureido)phenoxy)methyl)carbonyl-2-methyl-4-(4-fluorobenzyl)piperazine or a salt thereof, the organic acid and the adjuvant (preferably cellulose, cellulose derivatives, and lactose) are processed into pellets by means of extrusion and subsequent spheronization. The pellets that contain the active ingredient are then coated with the polymer matrix (preferably cellulose derivatives, acryl derivatives, vinyl polymers and shellac). Under certain circumstances, the active ingredient-containing pellets are coated with a subcoat (preferably cellulose derivatives and vinyl polymers) before the polymer matrix is applied. The function of the subcoat is the inhibition of incompatibilities between (2R)-1-((4-chloro-2-(ureido)phenoxy)methyl)carbonyl-2-methyl-4-(4-fluorobenzyl)piperazine or a salt thereof and the polymer matrix or a premature diffusion of (2R)-1-((4-chloro-2-(ureido)phenoxy)methyl)carbonyl-2-methyl-4-(4-fluorobenzyl)piperazine or a salt thereof in the polymer matrix during the storage of the pellets.

Another subject of the invention is a process for the production of a solid multiparticulate pharmaceutical agent formulation according to the invention, whereby (2R)-1-((4-chloro-2-(ureido)phenoxy)methyl)carbonyl-2-methyl-4-(4-fluorobenzyl)piperazine or a salt thereof, and the adjuvants (preferably cellulose, cellulose derivatives and lactose) are processed into pellets by means of extrusion and subsequent spheronization. The pellets that contain the active ingredient are then coated with the organic acid and the polymer matrix (preferably cellulose derivatives, acryl derivatives, vinyl polymers and shellac). Under certain circumstances, the active ingredient-containing pellets can be coated with a subcoat (preferably cellulose derivatives and vinyl polymers) before the polymer matrix is applied.

Another subject of the invention is a process for the production of a solid multiparticulate pharmaceutical agent formulation according to the invention, whereby (2R)-1-((4-chloro-2-(ureido)phenoxy)methyl)carbonyl-2-methyl-4-(4-fluorobenzyl)piperazine or a salt thereof, the polymer matrix, the organic acid and the adjuvant are processed into pellets by means of direct pelletization. In this case, the starting substances are mixed and processed into pellets by means of a binder solution (wet granulation) or melted additives (e.g., fats).

Another subject of the invention is a process for the production of a solid multiparticulate pharmaceutical agent formulation according to the invention, whereby (2R)-1-((4-chloro-2-(ureido)phenoxy)methyl)carbonyl-2-methyl-4-(4-fluorobenzyl)piperazine or a salt thereof, the polymer matrix, the organic acid and the adjuvant are processed into pellets by means of spray-drying or spray-solidification.

Another subject of the invention is a process for the production of a solid multiparticulate pharmaceutical agent formulation according to the invention, whereby (2R)-1-((4-chloro-2-(ureido)phenoxy)methyl)carbonyl-2-methyl-4-(4-fluorobenzyl)piperazine or a salt thereof, the polymer matrix, the organic acid and the adjuvant are processed into pellets by means of rotor granulation.

The invention also relates to a process for the production of a solid pharmaceutical agent formulation according to the invention, whereby the polymer matrix, the organic acid and the adjuvant are processed into pellets by the layered application onto (2R)-1-((4-chloro-2-(ureido)phenoxy)methyl)carbonyl-2-methyl-4-(4-fluorobenzyl)piperazine or a salt thereof (layering).

The invention also relates to a process for the production of a solid pharmaceutical agent formulation according to the invention, whereby (2R)-1-((4-chloro-2-(ureido)phenoxy)methyl)carbonyl-2-methyl-4-(4-fluorobenzyl)piperazine or a salt thereof, the polymer matrix, the organic acid and the adjuvant are processed into pellets by the layered application onto an active ingredient-free core (so-called non-pareils). In this process, (2R)-1-((4-chloro-2-(ureido)phenoxy)methyl)carbonyl-2-methyl-4-(4-fluorobenzyl)piperazine or a salt thereof is generally first applied onto an active ingredient-free core (so-called non-pareils). Then, the organic acid is applied. At the end of the process, the pellets are coated with a polymer matrix (preferably cellulose derivatives, acryl derivatives, vinyl polymers and shellac). Under certain circumstances, the pellets can be coated with a subcoat (preferably cellulose derivatives and vinyl polymers) before the polymer matrix is applied.

The invention also relates to a process for filling the pellets that are produced in capsules that are used pharmaceutically (preferably gelatin capsules, starch capsules or cellulose derivative capsules) or the pressing into tablets of pellets that are produced. The filling of pellets in capsules or processing of pellets into tablets can optionally be carried out with the addition of other adjuvants (preferably cellulose, cellulose derivatives, lactose, lubricants and flow-regulating agents).

The subject of the invention is also a process for the production of a solid pharmaceutical agent formulation according to the invention, whereby (2R)-1-((4-chloro-2-(ureido)phenoxy)methyl)carbonyl-2-methyl-4-(4-fluorobenzyl)piperazine or a salt thereof is mixed with the polymer matrix, the organic acid, the lubricant and the adjuvants and then is processed by means of direct tableting into minitablets (of a preferred tablet diameter of 1-5 mm).

In addition, the invention relates to a process for the production of a solid pharmaceutical agent formulation according to the invention, whereby (2R)-1-((4-chloro-2-(ureido)phenoxy)methyl)carbonyl-2-methyl-4-(4-fluorobenzyl)piperazine or a salt thereof, the polymer matrix, the organic acid and the adjuvant are subjected to an operation referred to as granulation before the mixing and tableting. After the granulation and the addition of the lubricant, the starting substances are processed into minitablets (of a preferred tablet diameter of 1-5 mm).

The invention also relates to a process for filling the minitablets that are produced in capsules that are used for pharmaceutical purposes (preferably gelatin capsules, starch capsules or cellulose-derivative capsules). The filling of minitablets in capsules can optionally be carried out with the addition of other adjuvants (preferably cellulose, cellulose derivatives, lactose).

The subject of the invention is also the use of the solid pharmaceutical agent formulation according to the invention for the production of a medication for treating inflammatory diseases. The inflammatory disease can be, e.g., multiple sclerosis, rheumatoid arthritis, psoriasis or atopic dermatitis. The treatment of a patient who suffers from an inflammatory disease is preferably carried out by administration of one tablet during the day.

### DESCRIPTION OF THE FIGURES

FIG. 1 describes the solubility of the piperazine urea-hydrogen sulfate based on the pH.

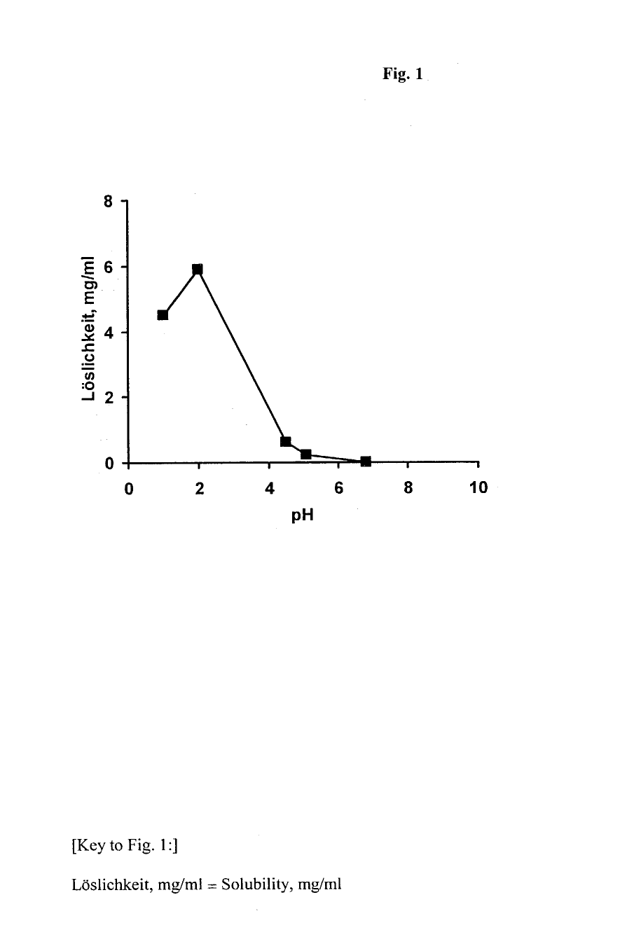

FIG. 2 shows the effects of the addition of SDS (sodium dodecyl sulfate) on the release of the piperazine urea-hydrogen sulfate in phosphate buffer solution, pH 6.8 (33% piperazine urea-hydrogen sulfate and 25% Kollidon SR®, relative to the total weight of the tablet).

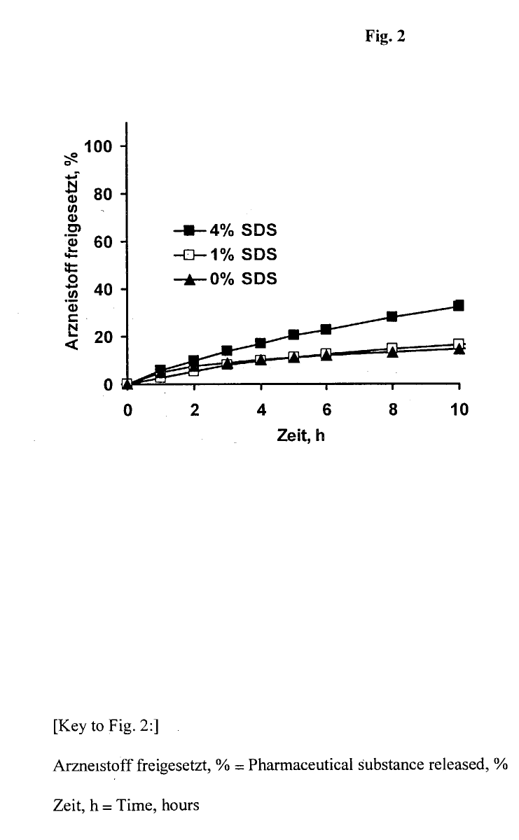

FIG. 3 shows the effect of fumaric acid (%, relative to the total weight of the tablet) on the release of the piperazine urea-hydrogen sulfate in phosphate buffer solution, pH 6.8 (33% piperazine urea-hydrogen sulfate and 25% Kollidon SR®, relative to the total weight of the tablet).

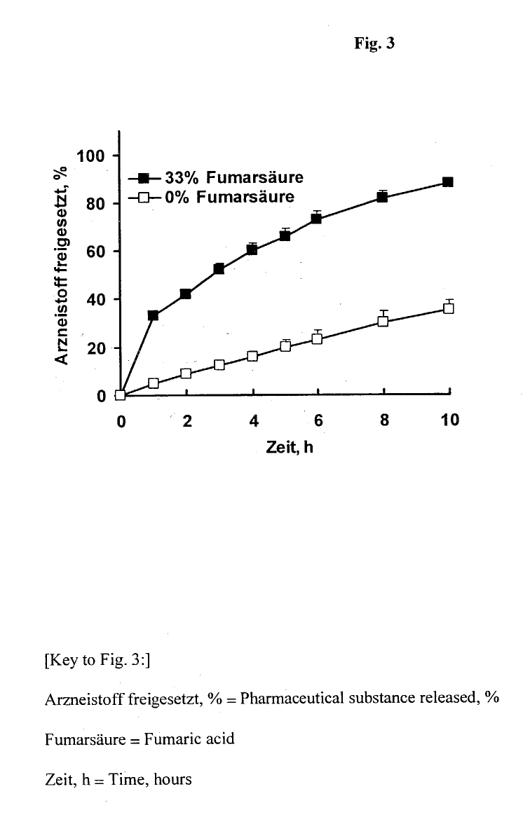

FIG. 4 shows the effect of the addition of different concentrations of fumaric acid (%, relative to the total weight of the tablet) on the release of piperazine urea-hydrogen sulfate in phosphate buffer solution, pH 6.8 (33% piperazine urea-hydrogen sulfate and 25% Kollidon SR®, relative to the total weight of the tablet).

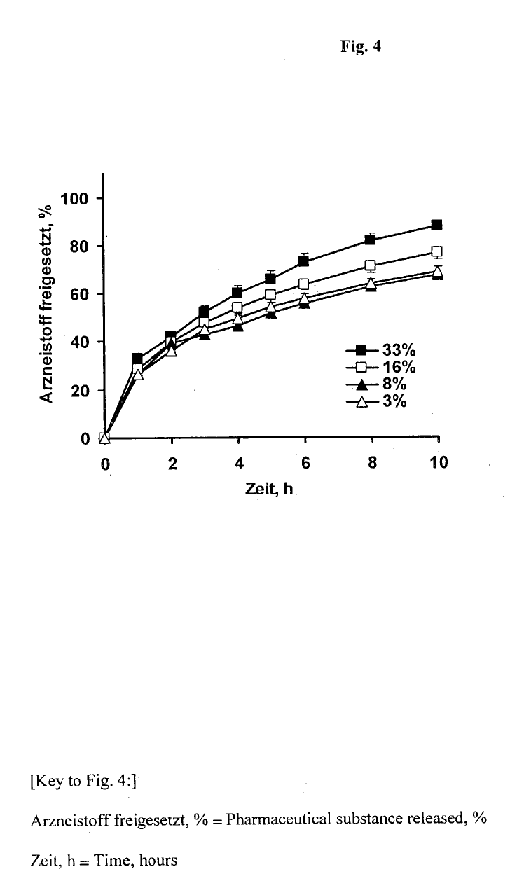

FIG. 5 shows the effect of the pH on the release of piperazine urea-hydrogen sulfate (33% piperazine urea-hydrogen sulfate, 25% Kollidon SR and 16% fumaric acid, relative to the total weight of the tablet).

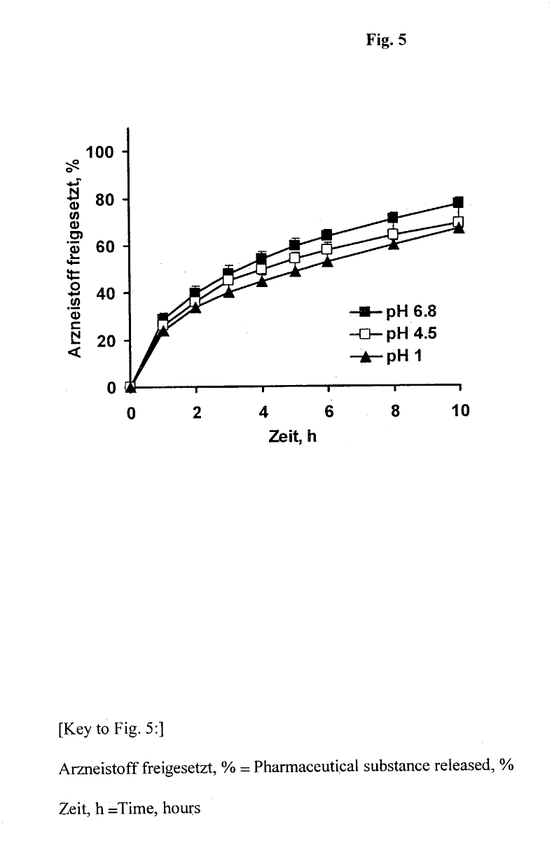

FIG. 6 shows the effect of the pH on the release of piperazine urea-hydrogen sulfate (33% piperazine urea-hydrogen sulfate, 12.5% Kollidon SR® and 16% fumaric acid, relative to the total weight of the tablet).

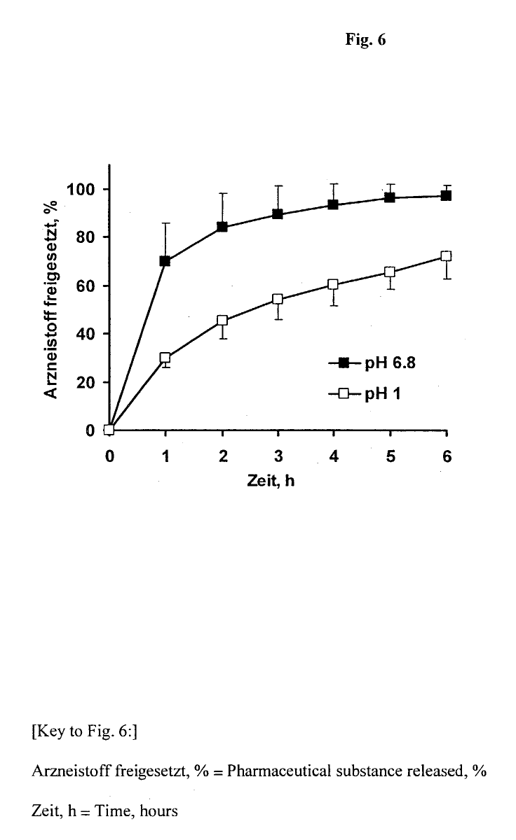

FIG. 7 shows the effect of the pH on the release of the piperazine urea-hydrogen sulfate (33% piperazine urea-hydrogen sulfate, 25% Kollidon SR®, 16% fumaric acid and 10% microcrystalline cellulose, relative to the total weight of the tablet).

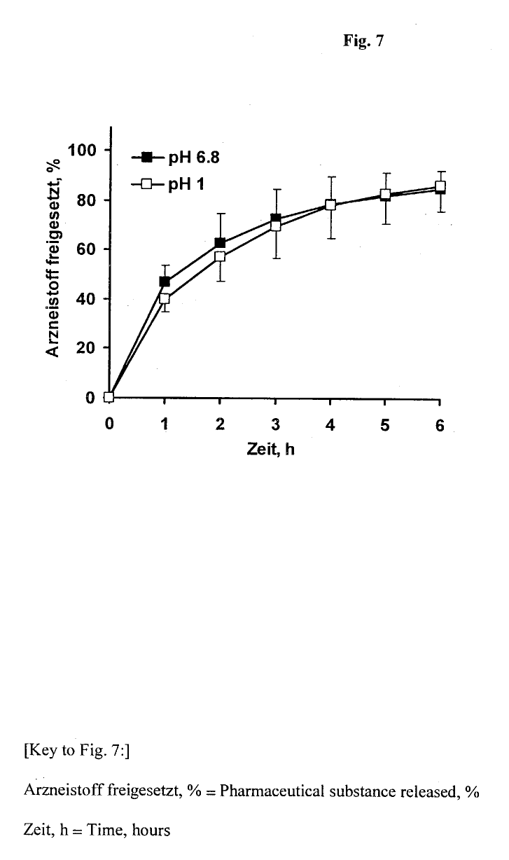

FIG. 8 shows the particle size distribution, determined by means of laser diffractometry, of a typical powder molding compound for direct tableting.

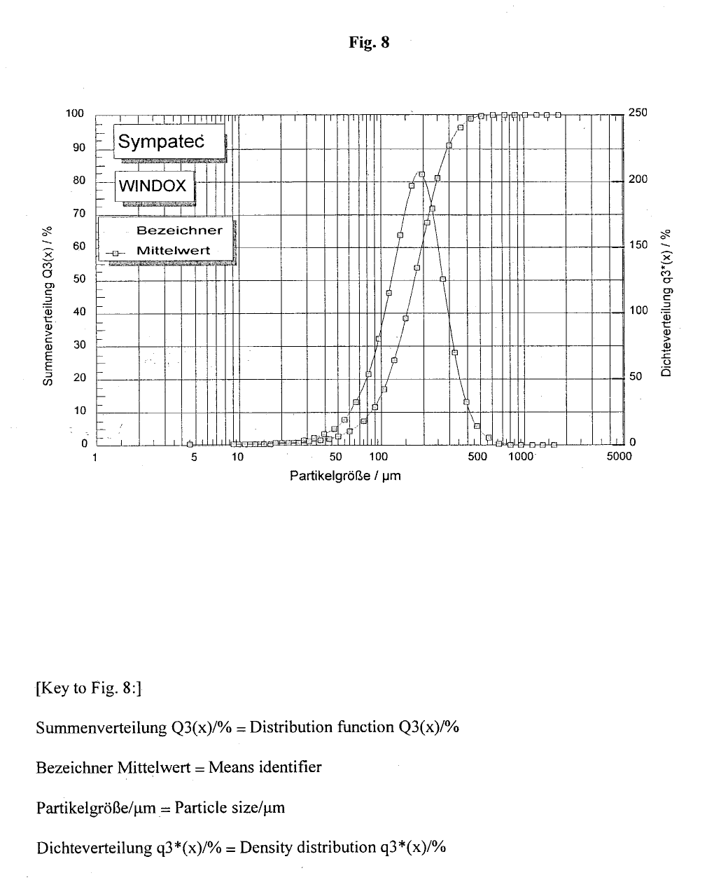

FIG. 9 shows the effect of the addition of different polymer matrices (Examples 3-9) on the release of the piperazine urea-hydrogen sulfate in phosphate buffer solution, pH 6.8.

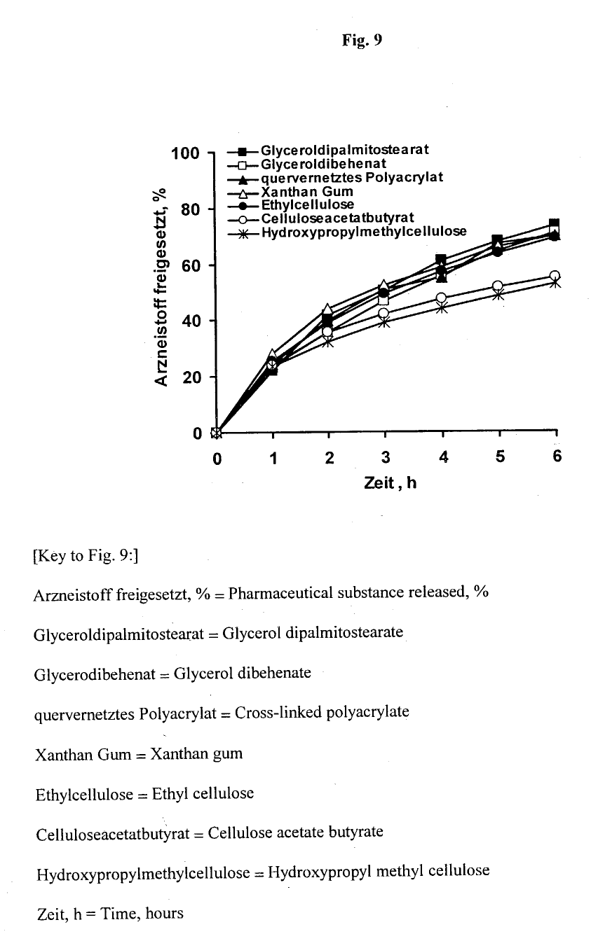

FIG. 10 shows the effect of the addition of different organic acids (Examples 10-13) on the release of the piperazine urea-hydrogen sulfate in phosphate buffer solution, pH 6.8.

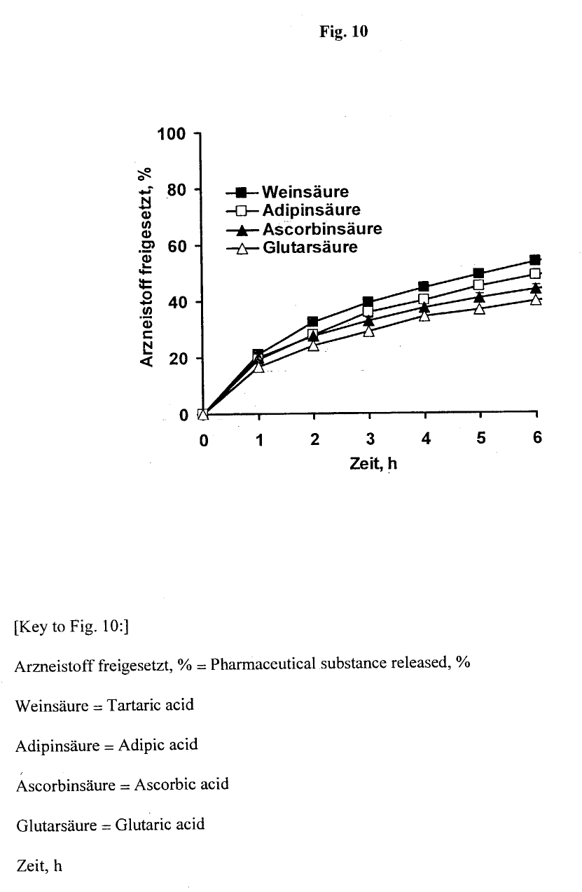

FIG. 11 shows, in semilogarithmic visualization, the effect of the pharmaceutical substance formulation on in-vivo plasma levels in humans after 100 mg of piperazine urea-hydrogen sulfate is administered in the form of a conventional oral formulation, as well as after formulations that are mentioned in Example 1 (matrix tablet C) and 2 (matrix tablet E) are administered.

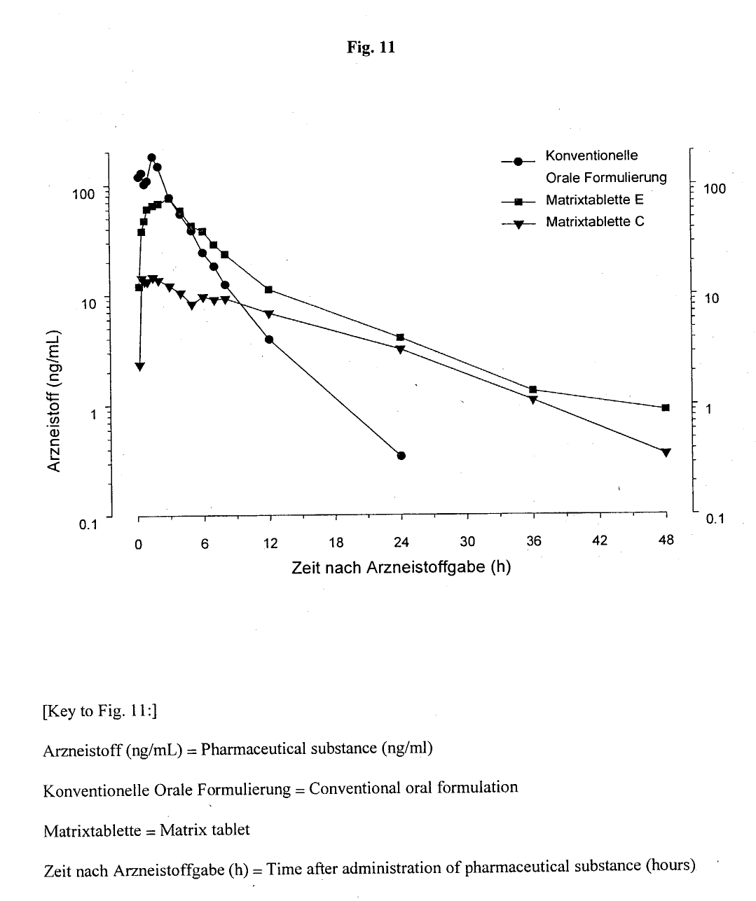

Without further elaboration, it is believed that one skilled in the art can, using the preceding description, utilize the present invention to its fullest extent. The following preferred specific embodiments are, therefore, to be construed as merely illustrative, and not limitative of the remainder of the disclosure in any way whatsoever. In the foregoing and in the following examples, all temperatures are set forth uncorrected in degrees Celsius and, all parts and percentages are by weight, unless otherwise indicated.

### EXAMPLE

### Example 1

Production of a Matrix Tablet by Means of Direct Tableting

Composition per basic unit:

100 mg of piperazine urea-hydrogen sulfate

69 mg of lactose

75 mg of Kollidon SR®

50 mg of fumaric acid

3 mg of highly dispersed silicon dioxide

3 mg of magnesium stearate

Lactose, piperazine urea-hydrogen sulfate and Kollidon SR® are individually sieved and mixed in the above-mentioned sequence in the turbula for 10 minutes. Fumaric acid, sieved, is added, and all components are mixed for another 5 minutes in the turbula. Highly dispersed silicon dioxide, sieved, is added, and all components are mixed for another 5 minutes in the turbula. Magnesium stearate, sieved, is spread on, and all components are mixed in the turbula for another 30 seconds. Tableting of the powder molding compound is then carried out by means of an eccentric tablet press or a rotary tablet press.

### Example 2

Production of a Matrix Tablet by Means of Direct Tableting

Composition per basic unit:

100 mg of piperazine urea-hydrogen sulfate

39 mg of lactose

75 mg of Kollidon SR®

50 mg of fumaric acid

30 mg of microcrystalline cellulose

3 mg of highly dispersed silicon dioxide

3 mg of magnesium stearate

Lactose, piperazine urea-hydrogen sulfate, Kollidon SR® and microcrystalline cellulose are sieved individually and mixed in the above-mentioned sequence in the turbula for 10 minutes. Fumaric acid, sieved, is added, and all components are mixed for another 5 minutes in the turbula. Highly dispersed silicon dioxide, sieved, is added, and all components are mixed for another 5 minutes in the turbula. Magnesium stearate, sieved, is spread on, and all components are mixed for another 30 seconds in the turbula. Tableting of the powder molding compound is then carried out by means of an eccentric tablet press or a rotary tablet press.

### Example 3

Production of a Matrix Tablet by Means of Direct Tableting

Composition per basic unit:

100 mg of piperazine urea-hydrogen sulfate

104 mg of lactose

40 mg of Precirol® ATO 5

50 mg of fumaric acid

3 mg of highly dispersed silicon dioxide

3 mg of magnesium stearate

Lactose, piperazine urea-hydrogen sulfate and Precirol® ATO 5 (glycerol dipalmitostearate) are individually sieved and mixed in the above-mentioned sequence in the turbula for 10 minutes. Fumaric acid, sieved, is added, and all components are mixed for another 5 minutes in the turbula. Highly dispersed silicon dioxide, sieved, is added, and all components are mixed for another 5 minutes in the turbula. Magnesium stearate, sieved, is spread on, and all components are mixed in the turbula for another 30 seconds. Tableting of the powder molding compound is then carried out by means of an eccentric tablet press or a rotary tablet press.

### Example 4

Production of a Matrix Tablet by Means of Direct Tableting

Composition per basic unit:

100 mg of piperazine urea-hydrogen sulfate

104 mg of lactose

40 mg of Compritol® 888 ATO

50 mg of fumaric acid

3 mg of highly dispersed silicon dioxide

3 mg of magnesium stearate

Lactose, piperazine urea-hydrogen sulfate and Compritrol® 888 ATO (glycerol dibehenate) are individually sieved and mixed in the above-mentioned sequence in the turbula for 10 minutes. Fumaric acid, sieved, is added, and all components are mixed for another 5 minutes in the turbula. Highly dispersed silicon dioxide, sieved, is added, and all components are mixed for another 5 minutes in the turbula. Magnesium stearate, sieved, is spread on, and all components are mixed in the turbula for another 30 seconds. Tableting of the powder molding compound is then carried out by means of an eccentric tablet press or a rotary tablet press.

### Example 5

Production of a Matrix Tablet by Means of Direct Tableting

Composition per basic unit:

100 mg of piperazine urea-hydrogen sulfate

69 mg of lactose

75 mg of Carbopol® 71 G

50 mg of fumaric acid

3 mg of highly dispersed silicon dioxide

3 mg of magnesium stearate

Lactose, piperazine urea-hydrogen sulfate and Carbopol 71 G® (cross-linked polyacrylate) are individually sieved and mixed in the above-mentioned sequence in the turbula for 10 minutes. Fumaric acid, sieved, is added, and all components are mixed for another 5 minutes in the turbula. Highly dispersed silicon dioxide, sieved, is added, and all components are mixed for another 5 minutes in the turbula. Magnesium stearate, sieved, is spread on, and all components are mixed in the turbula for another 30 seconds. Tableting of the powder molding compound is then carried out by means of an eccentric tablet press or a rotary tablet press.

### Example 6

Production of a Matrix Tablet by Means of Direct Tableting

Composition per basic unit:

100 mg of piperazine urea-hydrogen sulfate

69 mg of lactose

75 mg of Xantural® 75

50 mg of fumaric acid

3 mg of highly dispersed silicon dioxide

3 mg of magnesium stearate

Lactose, piperazine urea-hydrogen sulfate and Xantural® 75 (xanthan gum) are individually sieved and mixed in the above-mentioned sequence in the turbula for 10 minutes. Fumaric acid, sieved, is added, and all components are mixed for another 5 minutes in the turbula. Highly dispersed silicon dioxide, sieved, is added, and all components are mixed for another 5 minutes in the turbula. Magnesium stearate, sieved, is spread on, and all components are mixed in the turbula for another 30 seconds. Tableting of the powder molding compound is then carried out by means of an eccentric tablet press or a rotary tablet press.

### Example 7

Production of a Matrix Tablet by Means of Direct Tableting

Composition per basic unit:

100 mg of piperazine urea-hydrogen sulfate

84 mg of lactose

60 mg of ethylcellulose 100

50 mg of fumaric acid

3 mg of highly dispersed silicon dioxide

3 mg of magnesium stearate

Lactose, piperazine urea-hydrogen sulfate and ethylcellulose 100 are individually sieved and mixed in the above-mentioned sequence in the turbula for 10 minutes. Fumaric acid, sieved, is added, and all components are mixed for another 5 minutes in the turbula. Highly dispersed silicon dioxide, sieved, is added, and all components are mixed for another 5 minutes in the turbula. Magnesium stearate, sieved, is spread on, and all components are mixed in the turbula for another 30 seconds. Tableting of the powder molding compound is then carried out by means of an eccentric tablet press or a rotary tablet press.

### Example 8

Production of a Matrix Tablet by Means of Direct Tableting

Composition per basic unit:

100 mg of piperazine urea-hydrogen sulfate

10 mg of lactose

134 mg of cellulose acetate butyrate 171-15 PG

50 mg of fumaric acid

3 mg of highly dispersed silicon dioxide

3 mg of magnesium stearate

Lactose, piperazine urea-hydrogen sulfate and cellulose acetate butyrate 171-15 PG (cellulose acetate butyrate) are individually sieved and mixed in the above-mentioned sequence in the turbula for 10 minutes. Fumaric acid, sieved, is added, and all components are mixed for another 5 minutes in the turbula. Highly dispersed silicon dioxide, sieved, is added, and all components are mixed for another 5 minutes in the turbula. Magnesium stearate, sieved, is spread on, and all components are mixed in the turbula for another 30 seconds. Tableting of the powder molding compound is then carried out by means of an eccentric tablet press or a rotary tablet press.

### Example 9

Production of a Matrix Tablet by Means of Direct Tableting

Composition per basic unit:

100 mg of piperazine urea-hydrogen sulfate

94 mg of lactose

50 mg of hydroxypropyl methyl cellulose K 15 M

50 mg of fumaric-acid

3 mg of highly dispersed silicon dioxide

3 mg of magnesium stearate

Lactose, piperazine urea-hydrogen sulfate and hydroxypropyl methyl cellulose K 15 M are individually sieved and mixed in the above-mentioned sequence in the turbula for 10 minutes. Fumaric acid, sieved, is added, and all components are mixed for another 5 minutes in the turbula. Highly dispersed silicon dioxide, sieved, is added, and all components are mixed for another 5 minutes in the turbula. Magnesium stearate, sieved, is spread on, and all components are mixed in the turbula for another 30 seconds. Tableting of the powder molding compound is then carried out by means of an eccentric tablet press or a rotary tablet press.

### Example 10

Production of a Matrix Tablet by Means of Direct Tableting

Composition per basic unit:

100 mg of piperazine urea-hydrogen sulfate

69 mg of lactose

75 mg of Kollidon SR®

50 mg of glutaric acid

3 mg of highly dispersed silicon dioxide

3 mg of magnesium stearate

Lactose, piperazine urea-hydrogen sulfate and Kollidon SR® are individually sieved and mixed in the above-mentioned sequence in the turbula for 10 minutes. Glutaric acid, sieved, is added, and all components are mixed for another 5 minutes in the turbula. Highly dispersed silicon dioxide, sieved, is added, and all components are mixed for another 5 minutes in the turbula. Magnesium stearate, sieved, is spread on, and all components are mixed in the turbula for another 30 seconds. Tableting of the powder molding compound is then carried out by means of an eccentric tablet press or a rotary tablet press.

### Example 11

Production of a Matrix Tablet by Means of Direct Tableting

Composition per basic unit:

100 mg of piperazine urea-hydrogen sulfate

69 mg of lactose

75 mg of Kollidon SR®

50 mg of tartaric acid

3 mg of highly dispersed silicon dioxide

3 mg of magnesium stearate

Lactose, piperazine urea-hydrogen sulfate and Kollidon SR® are individually sieved and mixed in the above-mentioned sequence in the turbula for 10 minutes. Tartaric acid, sieved, is added, and all components are mixed for another 5 minutes in the turbula. Highly dispersed silicon dioxide, sieved, is added, and all components are mixed for another 5 minutes in the turbula. Magnesium stearate, sieved, is spread on, and all components are mixed in the turbula for another 30 seconds. Tableting of the powder molding compound is then carried out by means of an eccentric tablet press or a rotary tablet press.

### Example 12

Production of a Matrix Tablet by Means of Direct Tableting

Composition per basic unit:

100 mg of piperazine urea-hydrogen sulfate

69 mg of lactose

75 mg of Kollidon SR®

50 mg of adipic acid

3 mg of highly dispersed silicon dioxide

3 mg of magnesium stearate

Lactose, piperazine urea-hydrogen sulfate and Kollidon SR® are individually sieved and mixed in the above-mentioned sequence in the turbula for 10 minutes. Adipic acid, sieved, is added, and all components are mixed for another 5 minutes in the turbula. Highly dispersed silicon dioxide, sieved, is added, and all components are mixed for another 5 minutes in the turbula. Magnesium stearate, sieved, is spread on, and all components are mixed in the turbula for another 30 seconds. Tableting of the powder molding compound is then carried out by means of an eccentric tablet press or a rotary tablet press.

### Example 13

Production of a Matrix Tablet by Means of Direct Tableting

Composition per basic unit:

100 mg of piperazine urea-hydrogen sulfate

69 mg of lactose

75 mg of Kollidon SR®

50 mg of ascorbic acid

3 mg of highly dispersed silicon dioxide

3 mg of magnesium stearate

Lactose, piperazine urea-hydrogen sulfate and Kollidon SR® are individually sieved and mixed in the above-mentioned sequence in the turbula for 10 minutes. Ascorbic acid, sieved, is added, and all components are mixed for another 5 minutes in the turbula. Highly dispersed silicon dioxide, sieved, is added, and all components are mixed for another 5 minutes in the turbula. Magnesium stearate, sieved, is spread on, and all components are mixed in the turbula for another 30 seconds. Tableting of the powder molding compound is then carried out by means of an eccentric tablet press or a rotary tablet press.

### Example 14

Production of a Matrix Tablet by Means of Direct Tableting with Subsequent Film Coating

Composition per basic unit:

100 mg of piperazine urea-hydrogen sulfate

82.5 mg of lactose

60 mg of Kollidon SR®

50 mg of fumaric acid

3 mg of highly dispersed silicon dioxide

4.5 mg of magnesium stearate

7.6 mg of hydroxypropyl methyl cellulose, visc. 5

1.5 mg of talc

5.9 mg of titanium dioxide, E 171

0.02 mg of iron oxide pigment yellow, E 172 (EOP yellow)

Lactose, piperazine urea-hydrogen sulfate and Kollidon SR® are individually sieved and mixed in the above-mentioned sequence in the turbula for 10 minutes. Fumaric acid, sieved, is added, and all components are mixed for another 5 minutes in the turbula. Highly dispersed silicon dioxide, sieved, is added, and all components are mixed for another 5 minutes in the turbula. Magnesium stearate, sieved, is spread on, and all components are mixed in the turbula for another 30 seconds. Tableting of the powder molding compound is then carried out by means of an eccentric tablet press or a rotary tablet press (tablet cores). Talc, iron oxide pigment yellow and titanium dioxide are suspended in water (dye suspension) while being stirred (e.g., Ultra-Turrax mixer or colloid mill). Hydroxypropyl methyl cellulose is dissolved in water (binder solution) while being stirred (e.g., Ultra-Turrax mixer or colloid mill). Dye suspension and binder solution are combined (film coating) while being stirred (e.g., Ultra-Turrax mixer or colloid mill). The film coating that is produced is sprayed onto the tablet core in a drum coater while heat is supplied, whereby the water that is used evaporates.

### Example 15

Production of a Matrix Tablet by Means of Direct Tableting with Subsequent Film Coating

Composition per basic unit:

300 mg of piperazine urea-hydrogen sulfate

247.5 mg of lactose

180 mg of Kollidon SR®

150 mg of fumaric acid

9 mg of highly dispersed silicon dioxide

13.5 mg of magnesium stearate

10.1 mg of hydroxypropyl methyl cellulose, visc. 5

2 mg of talc

7.8 mg of titanium dioxide, E 171

0.03 mg of iron oxide pigment yellow, E 172 (EOP yellow)

Lactose, piperazine urea-hydrogen sulfate and Kollidon SR® are individually sieved and mixed in the above-mentioned sequence in the turbula for 10 minutes. Fumaric acid, sieved, is added, and all components are mixed for another 5 minutes in the turbula. Highly dispersed silicon dioxide, sieved, is added, and all components are mixed for another 5 minutes in the turbula. Magnesium stearate, sieved, is spread on, and all components are mixed in the turbula for another 30 seconds. Tableting of the powder molding compound is carried out by means of an eccentric tablet press or a rotary tablet press (tablet cores). Talc, iron oxide pigment yellow and titanium dioxide are suspended in water (dye suspension) while being stirred (e.g., Ultra-Turrax mixer or colloid mill). Hydroxypropyl methyl cellulose is dissolved in water (binder solution) while being stirred (e.g., Ultra-Turrax mixer or colloid mill). Dye suspension and binder solution are combined (film coating) while being stirred (e.g., Ultra-Turrax mixer or colloid mill). The film coating that is produced is sprayed onto the tablet core in a drum coater while heat is supplied, whereby the water that is used evaporates.

### Example 16

Production of Minitablets by Means of Direct Tableting

10 mg of piperazine urea-hydrogen sulfate

6.9 mg of lactose

7.5 mg of Kollidon SR®

5 mg of fumaric acid

0.3 mg of highly dispersed silicon dioxide

0.3 mg of magnesium stearate

Lactose, piperazine urea-hydrogen sulfate and Kollidon SR® are individually sieved and mixed in the above-mentioned sequence in the turbula for 10 minutes. Fumaric acid, sieved, is added, and all components are mixed for another 5 minutes in the turbula. Highly dispersed silicon dioxide, sieved, is added, and all components are mixed for another 5 minutes in the turbula. Magnesium stearate, sieved, is spread on, and all components are mixed in the turbula for another 30 seconds. Tableting of the powder molding compound into minitablets is then carried out by means of an eccentric tablet press or a rotary tablet press. The minitablets that are produced are delivered in hard-gelatin capsules.

### Example 17

Production of Minitablets with Subsequent Film Coating

Composition per basic unit:

10 mg of piperazine urea-hydrogen sulfate

8.25 mg of lactose

6 mg of Kollidon SR®

5 mg of fumaric acid

0.3 mg of highly dispersed silicon dioxide

0.45 mg of magnesium stearate

0.76 mg of hydroxypropyl methyl cellulose, visc. 5

0.15 mg of talc

0.59 mg of titanium dioxide, E 171

0.002 mg of iron oxide pigment yellow, E 172 (EOP yellow)

Lactose, piperazine urea-hydrogen sulfate and Kollidon SR® are individually sieved and mixed in the above-mentioned sequence in the turbula for 10 minutes. Fumaric acid, sieved, is added, and all components are mixed for another 5 minutes in the turbula. Highly dispersed silicon dioxide, sieved, is added, and all components are mixed for another 5 minutes in the turbula. Magnesium stearate, sieved, is spread on, and all components are mixed in the turbula for another 30 seconds. Tableting of the powder molding compound into minitablets is then carried out by means of an eccentric tablet press or a rotary tablet press (tablet cores). Talc, iron oxide pigment yellow and titanium dioxide are suspended in water (dye suspension) while being stirred (e.g., Ultra-Turrax mixer or colloid mill). Hydroxypropyl methyl cellulose is dissolved in water (binder solution) while being stirred (e.g., Ultra-Turrax mixer or colloid mill). Dye suspension and binder solution are combined (film coating) while being stirred (e.g., Ultra-Turrax mixer or colloid mill). The film coating that is produced is sprayed onto the tablet core in a drum coater while heat is supplied, whereby the water that is used evaporates. The minitablets that are produced are delivered in hard-gelatin capsules.

### Example 18

Production of a Matrix Tablet after Granulation

Composition per basic unit:

100 mg of piperazine urea-hydrogen sulfate

72 mg of lactose

75 mg of Kollidon SR®

50 mg of fumaric acid

3 mg of magnesium stearate

Lactose, piperazine urea-hydrogen sulfate, Kollidon SR® and fumaric acid are introduced into a fluidized-bed granulator and granulated while water is being sprayed. Magnesium stearate is spread on the dried granulate and mixed for 30 seconds in the turbula. The tableting of the granulate is then carried out by means of an eccentric tablet press or a rotary tablet press.

### Example 19

Production of Minitablets after Granulation

Composition per basic unit:

10 mg of piperazine urea-hydrogen sulfate

7.2 mg of lactose

7.5 mg of Kollidon SR®

5 mg of fumaric acid

0.3 mg of magnesium stearate

Lactose, piperazine urea-hydrogen sulfate, Kollidon SR® and fumaric acid are introduced into a fluidized-bed granulator and granulated while water is being sprayed. Magnesium stearate is spread on the dried granulate and mixed for 30 seconds in the turbula. The tableting of the granulate into minitablets is then carried out by means of an eccentric tablet press or a rotary tablet press. The minitablets that are produced are delivered in hard-gelatin capsules.

### Example 20

Production of Pellets by Means of Extrusion and Spheronization

Composition per capsule:

60 mg of piperazine urea-hydrogen sulfate

30 mg of microcrystalline cellulose

10 mg of fumaric acid

25.5 mg of Eudragit® NE 30 D

4.25 mg of talc

0.18 mg of anhydrous, highly-dispersed silicon dioxide

Piperazine urea-hydrogen sulfate, microcrystalline cellulose and fumaric acid are processed into pellets by means of a Nica-pelletizing system. In this process, first piperazine urea-hydrogen sulfate, microcrystalline cellulose and fumaric acid, in a dry state, are mixed. The powder mixture is then extruded with the addition of water. The processing of the extrudate into pellets is carried out with use of a spheronizer. An aqueous suspension that consists of Eudragit® NE 30 D and talc is sprayed onto the pellets while heat is being supplied by means of a fluidized-bed granulator using a Wurster. The delivery of the film-coated pellets in hard-gelatin capsules is carried out with the addition of silicon dioxide.

### Example 21

Measurement of the Release of Piperazine Urea-Hydrogen Sulfate

Measurement of the active ingredient release is carried out according to a one-compartment method (vane-stirrer apparatus), as described in U.S. Pharmacopeia USP XXIV. The release of the piperazine urea-hydrogen sulfate was examined at pH 1 (0.1 N hydrochloric acid) and in phosphate buffer solution, pH 4.5 and 6.8 (composition, see USP XXIV). To adjust sink conditions, which ensure that the release of piperazine urea-hydrogen sulfate is controlled primarily by the formulation, surfactant (SDS) or hydroxypropyl-β-cyclodextrin is added to the release medium, if necessary.

### Example 22

Measurement of the Particle Size

The particle size of piperazine urea-hydrogen sulfate, lactose, Kollidon SR®, fumaric acid, microcrystalline cellulose or the powder mixtures that are mentioned in Examples 1 to 9 was determined by means of laser diffractometry (Muller, R. H., Schuhmann, R., Teilchengröβenmessung in der Laborpraxis [Particle Size Measurement in Laboratory Practice], Wissenschaftliche Verlagsgesellschaft mbH, Stuttgart, 1996). As measuring parameters, the volume distribution of the particle sizes was used.

The entire disclosures of all applications, patents and publications, cited herein and of corresponding German Application No. 101 52 351.3 filed Oct. 18, 2001, and U.S. Provisional Application Serial No. 60/330,410, filed Oct. 22, 2001 are incorporated by reference herein.

The preceding examples can be repeated with similar success by substituting the generically or specifically described reactants and/or operating conditions of this invention for those used in the preceding examples.

From the foregoing description, one skilled in the art can easily ascertain the essential characteristics of this invention and, without departing from the spirit and scope thereof, can make various changes and modifications of the invention to adapt it to various usages and conditions.

1. Solid pharmaceutical agent formulation that contains (2R)-1-((4-chloro-2-(ureido)phenoxy)methyl)carbonyl-2-methyl-4-(4-fluorobenzyl)piperazine or a salt thereof, characterized in that in addition, it contains a polymer matrix, an organic acid, a lubricant and one or more adjuvants and in that the particle sizes of the powder mixtures are up to 90% in the range between 0.1 and 750 μm.

2. Solid pharmaceutical agent formulation according to claim 1, whereby the polymer matrix is selected from the group that consists of cellulose derivatives, acryl derivatives, vinyl polymers, polyanhydrides, polyester polyorthoesters, polyurethanes, polycarbonates, polyphosphazenes, polyacetals, polysaccharides, sugar esters, diethylene glycol-monoethyl ethers, diethylene glycol monopalmitostearate, ethylene glycol-monopalmitostearate, glycerol behenates and glycerol dibehenates, glycerol distearates and glycerol palmitostearates, glycerol-monooleate 40, glycerol-monostearate 40-55, macrogolglycerol-laurates, macrogolglycerol-stearates, propylene glycol-monopalmitostearate, chitosan, galactomannan, pectin, shellac and alginates.

3. Solid pharmaceutical agent formulation according to claim 1 or 2, whereby the polymer matrix consists of a mixture of water-soluble polyvinyl pyrrolidone and water-insoluble polyvinyl acetate.

4. Solid pharmaceutical agent formulation according to one of claims 1-3, whereby the polymer matrix has the following composition: 80% polyvinyl acetate, 19% polyvinyl pyrrolidone, 0.8% sodium lauryl sulfate and 0.2% silicon dioxide.

5. Solid pharmaceutical agent formulation according to one of claims 1-4, whereby the organic acid is selected from the group that consists of fumaric acid, citric acid, tri-sodium citrate, Na-hydrogen citrate, ascorbic acid, maleic acid, maleic acid anhydride, tartaric acid, adipic acid, Na-hydrogen phosphate, succinic acid, glutaric acid, glutaric acid anhydride, potassium sorbate and sorbic acid.

6. Solid pharmaceutical agent formulation according to one of claims 1-5, whereby in addition a lubricant is added.

7. Solid pharmaceutical agent formulation according to one of claims 1-6, whereby the adjuvant is lactose, calcium diphosphate, mannitol or a starch.

8. Solid pharmaceutical agent formulation according to one of claims 1-7, whereby it contains microcrystalline cellulose as an additional adjuvant.

9. Solid pharmaceutical agent formulation according to one of claims 1-8, whereby in addition, it contains a flow-regulating agent.

10. Solid pharmaceutical agent formulation according to one of claims 1-9, whereby the particle sizes of the powder mixtures are up to 90% in the range between 20 and 400 μm.

11. Solid pharmaceutical agent formulation according to one of claims 1-10, whereby (2R)-1-((4-chloro-2-(ureido)phenoxy)methyl)carbonyl-2-methyl-4-(4-fluorobenzyl)piperazine or a salt thereof is dispersed homogeneously in the matrix.

12. Solid pharmaceutical agent formulation according to one of claims 1-11, whereby (2R)-1-((4-chloro-2-(ureido)phenoxy)methyl)carbonyl-2-methyl-4-(4-fluorobenzyl)piperazine or a salt thereof is surrounded by the matrix.

13. Process for the production of a solid pharmaceutical agent formulation according to one of claims 1-12, whereby (2R)-1-((4-chloro-2-(ureido)phenoxy)methyl)carbonyl-2-methyl-4-(4-fluorobenzyl)piperazine or a salt thereof is mixed with one or more adjuvants, the polymer matrix, the organic acid and the lubricant, and is put into tablet form, whereby all substances are present in powder form and are classified either individually before the mixing or together after the mixing.

14. Process for the production of a solid pharmaceutical agent formulation according to one of claims 1-12, whereby (2R)-1-((4-chloro-2-(ureido)phenoxy)methyl)carbonyl-2-methyl-4-(4-fluorobenzyl)piperazine or a salt thereof is mixed with one or more adjuvants, the polymer matrix and the organic acid and is then granulated, then the lubricant is added, and then it is put into tablet form.

15. Process for the production of a solid, multiparticulate pharmaceutical agent formulation according to one of claims 1-12, whereby (2R)-1-((4-chloro-2-(ureido)phenoxy)methyl)carbonyl-2-methyl-4-(4-fluorobenzyl)piperazine or a salt thereof, the polymer matrix, the organic acid and the adjuvant are processed into pellets by means of extrusion and subsequent spheronization.

16. Process for the production of a solid, multiparticulate pharmaceutical agent formulation according to one of claims 1-12, whereby (2R)-1-((4-chloro-2-(ureido)phenoxy)methyl)carbonyl-2-methyl-4-(4-fluorobenzyl)piperazine or a salt thereof, the polymer matrix, the organic acid and the adjuvant are mixed and are processed into pellets by means of a binder solution or melted additives.

17. Process for the production of a solid, multiparticulate pharmaceutical agent formulation according to one of claims 1-12, whereby (2R)-1-((4-chloro-2-(ureido)phenoxy)methyl)carbonyl-2-methyl-4-(4-fluorobenzyl)piperazine or a salt thereof, the polymer matrix, the organic acid and the adjuvant are processed into pellets by means of spray-drying or spray-solidification.

18. Process for the production of a solid, multiparticulate pharmaceutical agent formulation according to one of claims 1-12, whereby (2R)-1-((4-chloro-2-(ureido)phenoxy)methyl)carbonyl-2-methyl-4-(4-fluorobenzyl)piperazine or a salt thereof, the polymer matrix, the organic acid and the adjuvant are processed into pellets by means of rotor granulation.

19. Process for the production of a solid pharmaceutical agent formulation according to one of claims 1-12, whereby the polymer matrix, the organic acid and the adjuvant are processed into pellets by the layered application onto (2R)-1-((4-chloro-2-(ureido)phenoxy)methyl)carbonyl-2-methyl-4-(4-fluorobenzyl)piperazine or a salt thereof.

20. Use of a solid pharmaceutical agent formulation according to one of claims 1-12 for the production of a medication for treating inflammatory diseases.

21. Use according to claim 20, whereby the inflammatory disease is multiple sclerosis.

22. Use according to claim 20, whereby the inflammatory disease is rheumatoid arthritis.

23. Use according to claim 20, whereby the disease is psoriasis.

24. Use according to claim 20, whereby the disease is atopic dermatitis.
